(19) United States
(12) Patent Application Publication
(10) Pub. No.: US 2021/0363561 A1
(43) Pub. Date: Nov. 25, 2021

(54) METHOD OF ENRICHMENT OF MICRO-ORGANISMS IN WHOLE BLOOD

(71) Applicant: SIEMEMS HEAALTHCARE DIAGNOSTICS INC., Tarrytown, NY (US)

(72) Inventors: Nivedita Mitra, Bangalore (IN); Ramya Vutukuru, Bangalore (IN)

(21) Appl. No.: 17/255,507

(22) PCT Filed: Sep. 13, 2018
(86) PCT No.: PCT/EP2018/074753

§ 371 Date: Dec. 23, 2020

(30) Foreign Application Priority Data
Sep. 14, 2017 (EP) 17190994.8

(51) Int. Cl. C12Q 1/04; C12Q 1/24

(52) U.S. Cl. CPC C12Q 1/04; C12Q 1/24

(57) ABSTRACT

A method of enriching micro-organisms, in a metagenomics workflow, is disclosed. In an aspect of the disclosure, the method includes obtaining the sample suspected to contain micro-organisms; concentrating the micro-organisms from the sample; and processing the micro-organisms to obtain genetic information specific to the micro-organisms. In another aspect, for concentrating the micro-organisms from the sample, the method includes separating plasma from the whole blood sample; selectively removing white blood cells from the plasma; and isolating the micro-organisms from the plasma after selectively removing the white blood cells.

The present patent document is a § 371 nationalization of PCT Application Serial No. PCT/EP2018/074753, filed Sep. 13, 2018, designating the United States, which is hereby incorporated by reference, and this patent document also claims the benefit of European Patent Application No. 17190994.8, filed Sep. 14, 2017, which is also hereby incorporated by reference.

### TECHNICAL FIELD

The present disclosure relates to a method of enrichment of micro-organisms in whole blood.

### BACKGROUND

The metagenomics approach of nucleic acid sequencing is an unbiased way to analyze all the nucleic acid present in a sample. In comparison to pre-determined assay based methods of identification of pathogens, the metagenomics approach allows for identification of unsuspected micro-organisms. This enables further analysis of such pathogens which are not detected by traditional genome amplification approaches. The metagenomics approach also allows for the detection of antimicrobial resistance patterns that are distributed throughout the genome length. The presence of an overwhelming amount of human/host genomic deoxyribonucleic acid (DNA) background along with the target microbial DNA is the biggest technical challenge faced in the implementation of next generation sequencing workflows for identification of micro-organisms and their associated antibiotic resistance information from samples such as, but not limited to, blood, sputum, swab, and cerebrospinal fluid.

In a metagenomics study conducted on nasopharyngeal aspirates, disclosed in Yang et. al, J Clinical Microbiology, 49:3463-3469, it was revealed that up to 95% of raw next generation sequencing reads were that of human genomic DNA. In a sample like whole blood, this problem is bigger. The human genomic DNA present in the sample adds to sequencing time and cost. In addition, the subtraction of human genomic DNA component from that of the target-pathogen DNA is a computationally intensive process. There are multiple enrichment methods and kits available that may selectively eliminate human genomic DNA or selectively enrich pathogens. However, when such methods are used in a metagenomics workflow, adequate removal of human genomic DNA is not assured, thereby resulting in carry-over of significant amount of human genomic DNA. Such carry-over human genomic DNA may be a hindrance when very low-copies of pathogens are to be detected in the sample. If the workflow involves whole genomic amplification methods, there is a high vulnerability of amplification of non-specific DNA. This makes the downstream sequencing and bioinformatics lengthy and cumbersome.

In light of the above, there exists a need to provide a method that removes eukaryotic cells from a sample so as to make a more enriched pathogen sample available for the metagenomics workflow. There also exists a need to provide a method of removal of eukaryotic cells from a sample, which complements the current methods of DNA sequencing.

### SUMMARY AND DESCRIPTION

The object of the disclosure is therefore to provide a method for enrichment of micro-organisms in whole blood, thereby rendering an enriched pathogen sample for the metagenomics workflow of genomic sequencing.

The disclosure refers to a method of enrichment of micro-organisms in whole blood. The method includes obtaining a whole blood sample suspected to contain micro-organisms. The sample may be obtained, for example, from a patient. The method further includes concentrating the micro-organisms present in the sample. Concentrating micro-organisms enables removal of eukaryotic DNA background and efficient analysis of the micro-organisms. In a further act, the method includes processing the micro-organisms to identify genetic information pertaining to the micro-organisms. Obtaining genetic information of the micro-organisms enables determining, for example, the antibiotic resistance profile of the micro-organisms.

In an embodiment, in concentrating the micro-organisms from the sample, the method further includes separating plasma from the whole blood. Whole blood includes of red blood cells, white blood cells, platelets, and blood plasma. On separation of blood plasma, most of the red blood cells and white blood cells are removed, thereby enabling depletion of human genomic DNA from the sample. Therefore, the plasma may contain micro-organisms and remaining white blood cells. An advantage of separation of blood plasma is that the eukaryotic DNA background is reduced significantly. Therefore, interferences in analysis of micro-organisms and cellular components of the micro-organisms are decreased. The method further includes selectively removing the remaining white blood cells from the plasma. The plasma is therefore left with micro-organisms after the selective removal of white blood cells. Advantageously, the selective removal of white blood cells does not affect the micro-organisms present in the sample. Therefore, the micro-organisms remain intact while further depletion of the eukaryotic cells is achieved. The method further includes isolating the micro-organisms from the plasma. On removal of the eukaryotic cells, the plasma contains micro-organisms which are isolated for further processing. Isolating the micro-organisms from the plasma enables obtaining a concentrated sample of the micro-organisms.

According to an embodiment, the plasma is separated from the whole blood sample using centrifugation method. The whole blood sample may be subjected to plasma fractionation. On centrifugation, red blood cells and other blood components settle at the bottom of the centrifugation tube, thereby forming a buffy coat as a top layer. The separated plasma may be collected from above the layer of red blood cells. Centrifugation enables removal of most of the eukaryotic cells such as red blood cells as well as most of the white blood cells.

According to another embodiment, the remaining white blood cells are selectively removed from the plasma by incubating the plasma with at least one matrix coated with a primary substrate. The primary substrate may have affinity for white blood cells. Therefore, when the plasma containing white blood cells is brought in contact with the matrix coated with primary substrate, the white blood cells may bind to the primary substrate thereby forming a complex. The complex may be separated from the plasma so as to remove white blood cells from the plasma. Therefore, further depletion of eukaryotic cells from the sample is achieved

According to a further embodiment, the method may further include selective removal of white blood cells in the plasma using at least one matrix coated with secondary substrate. The secondary substrate may also have affinity for white blood cells. The matrix coated with secondary substrate may be used advantageously to remove any white blood cells that may be present in the plasma after selective removal with a matrix coated with a primary substrate. When the plasma containing white blood cells is brought in contact with the matrix coated with secondary substrate, the white blood cells may bind to the secondary substrate thereby forming a complex. The complex may be separated from the plasma so as to remove white blood cells from the plasma. Selectively removing white blood cells from the sample is a negative enrichment method. A negative enrichment method involves capturing and/or non-target cells such that target cells in the sample are intact and unaffected.

According to an embodiment, the micro-organisms are isolated from the plasma by incubating the plasma with at least one matrix coated with a tertiary substrate. Isolation of micro-organisms from the sample is a positive enrichment method. A positive enrichment method involves isolation of target cells from a given sample, leaving behind the non-target cells and cellular components in the sample. The tertiary substrate may have affinity for the micro-organisms. When the plasma containing micro-organisms is brought in contact with the matrix coated with tertiary substrate, the micro-organisms may bind to the tertiary substrate thereby forming a complex. The complex may be separated from the plasma so as to isolate the micro-organisms from the plasma. Therefore, on isolation of the micro-organisms using the tertiary substrate, a concentrated sample of micro-organisms is obtained. Thus, maximum depletion of eukaryotic cellular background is achieved.

According to another embodiment, the whole blood sample is centrifuged at a relative centrifugal force (RCF) ranging between 50×g and 500×g, and particularly between 100×g and 300×g. Centrifuging the whole blood sample at this RCF range advantageously allows for least human genomic DNA carry-over in the separated plasma.

According to an embodiment, the matrix for selective removal of white blood cells and for isolation of micro-organisms from the plasma may be chosen from a group that includes polystyrene beads and magnetic beads. The magnetic bead includes at least one particle of ferromagnetic, ferrimagnetic, super-magnetic or paramagnetic material. The surface of the matrix is adsorptive and is coated with at least one substrate. The substrate may have affinity for either white blood cells or for micro-organisms. Spherical beads provide a greater surface area for binding of the desired entity to the coated substrate.

According to an embodiment, the primary substrate and the secondary substrate are chosen from a group including CD45, CD15, CD14, CD3, CD4, CD8 and CD19 proteins. CD proteins are used as markers for immunophenotyping, thereby enabling cells to be differentiated based on what molecules are present on their surface. The CD proteins have a high affinity for white blood cells. Therefore, when a white blood cell in plasma is brought in contact with a matrix coated with a CD protein, a complex is formed between the white blood cell and the CD protein. Formation of complex enables efficient separation of such eukaryotic cells from the plasma.

According to an embodiment, the primary substrate and the secondary substrate are different. For example, if the primary substrate is a CD15 protein, the secondary substrate may be a CD45 protein.

According to an embodiment, the tertiary substrate is a pathogen binding protein and may be, for example, Apolipoprotein H, histone proteins, methyl-CpG-binding domains (MBD Proteins) or immune proteins. The pathogen binding proteins have a high affinity for micro-organisms. Therefore, when a matrix coated with the tertiary substrate is brought in contact with the sample, the micro-organisms bind to the substrate and for a complex. Use of a tertiary substrate allows for microbial isolation from the sample, thereby providing removal of complete eukaryotic DNA background.

According to another embodiment, the white blood cells bound to the matrix are separated from the plasma using a magnet. If the matrix onto which the substrates are coated is a magnetic bead, the magnet attracts the magnetic bead, thereby separating the complex from the plasma easily.

According to yet another embodiment, the white blood cells bound to the matrix are separated from the plasma using centrifugation. If the matrix onto which the substrates are coated is a polystyrene bead, the complex formed may be separated from the plasma using centrifugation. On centrifugation of the sample, the polystyrene beads along with the bound eukaryotic cells form a pellet at the bottom of the centrifuge tube. As the micro-organisms remain in the supernatant formed as a result of centrifugation, elimination of the white blood cells from the sample is achieved.

The present disclosure also provides for a kit for carrying out the abovementioned method. The kit includes one or more matrices coated with a primary substrate and a secondary substrate for removal of eukaryotic cells from the sample. The kit further includes one or more matrices coated with a tertiary substrate for isolation of micro-organisms from the sample, which may be used for the enrichment of micro organisms present in a sample.

### BRIEF DESCRIPTION OF THE DRAWINGS

The present disclosure is further described hereinafter with reference to illustrated embodiments shown in the accompanying drawings, in which:

FIG. 1 illustrates a schematic diagram of a flow chart of an embodiment of a method.

FIG. 2 illustrates a graph depicting an example of the effect of centrifugal speed on the human genomic DNA carry-over in the plasma.

FIG. 3 illustrates a graph depicting an example of the change in the amounts of human genomic DNA to microbial DNA with the employment of enrichment acts.

### DETAILED DESCRIPTION

Hereinafter, embodiments for carrying out the present disclosure are described in detail. The various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practiced without these specific details.

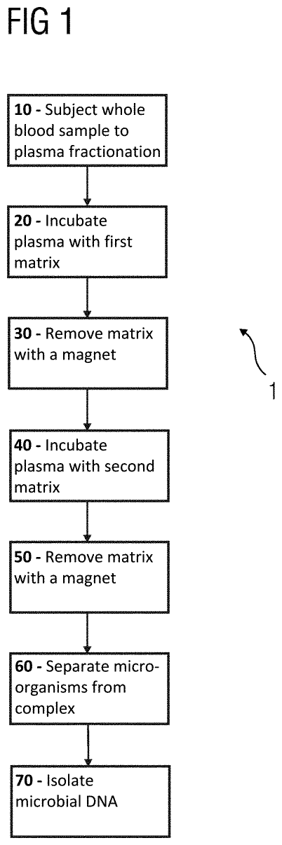

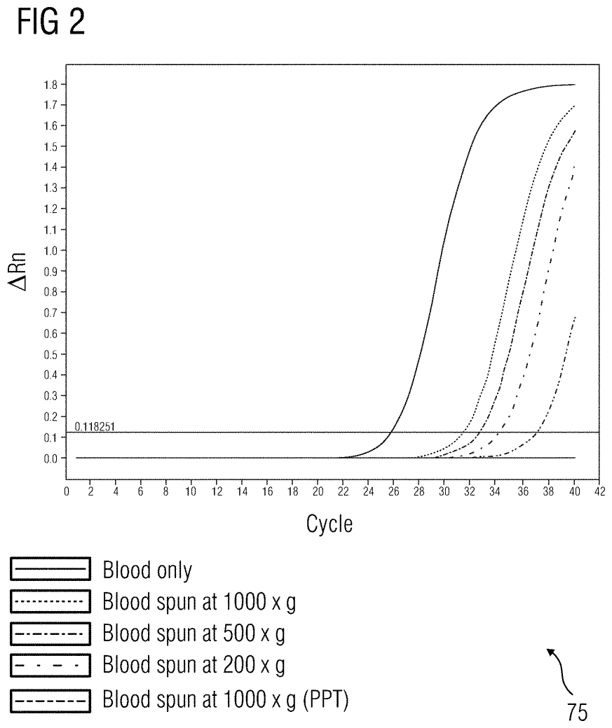

FIG. 1 illustrates a schematic representation of a flowchart of a method 1 for enrichment of micro-organisms. The sample used in the present embodiment is a whole blood sample suspected to contain micro-organisms. Alternatively, other fluids of the human body that are known to be used for such analyses may also be used. The sample volume may be in the range between 8 milliliter and 15 milliliter. In the present embodiment, a sample volume of 8 milliliter is considered for enrichment. In the first act 10 of the method 1, the whole blood sample suspected to contain micro-organisms is subject to plasma fractionation. The plasma is separated from the whole blood using centrifugation. 8 milliliter of whole blood sample is centrifuged at a relative centrifugal force (RCF) ranging between 50×g and 500×g, or between 150×g and 250×g for a time period in the range between 10 minutes to 30 minutes or between 15 minutes to 20 minutes. The relative centrifugal force used for separation of plasma is approximately one fifth of the recommended usual speed for plasma fractionation. At higher speeds, the quantity of human genomic DNA observed in the plasma is higher. This may be because of shearing of eukaryotic cells at higher speeds, resulting in release of human genomic DNA into the plasma. A graph 75 depicting the effect of centrifugal speed on the human genomic DNA carry-over in the plasma is illustrated in FIG. 2. The X-axis of the graph 75 represents the number of cycles of polymerase chain reaction for amplification of DNA present in the sample. The Y-axis of the graph 75 represents the amount of fluorescence produced and detected as a result of amplification of nucleic acids. The amount of fluorescence generated is directly proportional to the amount of DNA present in the sample being amplified. Therefore, as the polymerase chain reaction progresses, the amount of nucleic acids in the sample increases. If the amount of nucleic acid present in the sample is low, more number of cycles of polymerase chain reaction would be required to produce sufficient amount of amplified DNA. Therefore, greater number of amplification cycles would be required for generation of fluorescence beyond a certain threshold. In the present embodiment, the threshold of fluorescence is 0.118251. According to the graph 75, when a 50 micro liter sample containing only blood is amplified, the number of amplification cycles required to produce fluorescence above the threshold value is 25.62. 8 milliliter whole blood sample is spun at an RCF value of 1000×g to obtain plasma. A 50 micro liter representative sample of the obtained plasma is used for a quantitative DNA analysis. The 50 micro liter plasma sample is subjected to amplification. The number of amplification cycles required to produce fluorescence above the threshold value is 31.2. Further, an 8 milliliter whole blood sample is spun at an RCF value of 500×g to obtain plasma. When a representative sample amount of 50 micro liter of the obtained plasma is subjected to amplification, the number of amplification cycles required to produce fluorescence above the threshold value is 32.46. Similarly, an 8 milliliter whole blood sample is spun at an RCF value of 200×g to obtain plasma. When a representative sample amount of 50 micro liter of the obtained plasma is amplified, fluorescence is generated after 34.04 amplification cycles. The increase in the number of amplification cycles with reduction in the centrifugal speed is due to reduction in the amount of human genomic DNA in the plasma sample. At a higher centrifugal speed, cell shearing may occur, thereby allowing spilling of human genomic DNA from the blood cells into the sample. Therefore, spinning the sample at lower speeds reduces the risk of cell shearing and therefore reduces the amount of human genomic DNA in the sample.

At centrifugal speed of 200×g, a buffy coat is formed in the centrifuge tube, and plasma is collected from the top layer. Centrifugation of the whole blood sample at the range of relative centrifugal force enables 1000 fold depletion of human genomic DNA from eukaryotic cells such as red blood cells, platelets, and white blood cells. In the second act 20 of the method 1, the separated plasma is treated further such that any white blood cells remaining in the plasma are removed. Therefore, at act 20, the plasma is incubated with at least one matrix coated with a primary substrate. In the present embodiment, the at least one matrix is a magnetic bead. The primary substrate to be coated over the magnetic bead is chosen from a group of substrates that have affinity for white blood cells. In the present embodiment, the primary substrate is a CD45 protein. The plasma is incubated with the at least one matrix for a time period ranging between 1 minute to 60 minutes or between 10 minutes to 30 minutes. On incubation of the plasma with the CD45 proteins, the white blood cells bind to the CD45 proteins to form a complex. CD45 proteins selectively bind to any white blood cells that are present in the plasma. Once the complex is formed, at act 30, the matrix is removed using a magnet. Magnetic beads are attracted to a magnet, thereby enabling easy removal of the complex. In an embodiment, the plasma is further incubated with at least one matrix coated with a secondary substrate. Such secondary substrate is chosen from a group of substrates having affinity for white blood cells. In the present embodiment, the secondary substrate is CD15 protein. CD15 proteins selectively bind to any white blood cells that are remaining in the plasma, thereby forming a complex. Once the complex is formed, the matrix is removed using a magnet. At the end of the second act, 10,000 fold removal of human genomic DNA is achieved. This corresponds to removal of 99.99% of human genomic DNA.

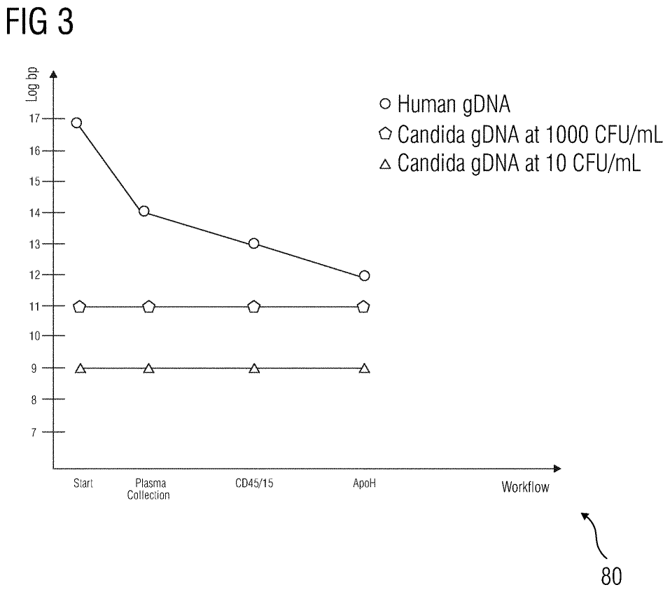

At act 40, the plasma is incubated with at least one matrix coated that is coated with a tertiary substrate. In the present embodiment, the matrix is a magnetic bead. Such tertiary substrate is chosen from a group of substrates that have an affinity for microbial cells. In the present embodiment, the tertiary substrate is Apolipoprotein H (ApoH) from ApoH Technologies®. ApoH proteins are plasmatic proteins that are capable of binding to micro-organisms that may be present in a given sample. The plasma is incubated with the at least one matrix for a time period ranging between 1 minute to 60 minutes or between 10 minutes to 30 minutes. On incubation of the plasma with the ApoH proteins, the micro-organisms bind to the ApoH proteins to form a complex. Once the complex is formed, at act 50, the matrix is removed using a magnet. Magnetic beads are attracted to a magnet, thereby enabling easy removal of the complex. This positive enrichment method for isolating the micro-organisms enables complete removal of human genomic DNA, thereby resulting in 10⁴ human genomic DNA base pairs per base pair of microbial DNA. A reduction in the amount of human genomic DNA to microbial DNA at each act of the method is illustrated as a graphical representation 80 in FIG. 3. A 100,000 fold depletion of human genomic DNA is achieved through the abovementioned method acts. The loss of microbial cells is minimal in comparison to depletion of eukaryotic cells. At act 60, the micro-organisms may be separated from the complex for further downstream processing at act 70, such as lysis of microbial cells for isolation of microbial DNA. The downstream processing may also include amplification of the genomic DNA of the micro-organisms and DNA sequencing.

The instant teachings also provide kits designed to expedite performance of the subject methods. Kits serve to expedite the performance of the methods of interest by assembling two or more components required for carrying out the disclosed methods. Kits may contain components in pre-measured unit amounts to minimize the need for measurements by end-users. Kits may include instructions for performing one or more of the disclosed methods. The kit components may be optimized to operate in conjunction with one another.

In an embodiment, kits include primary substrate and secondary substrate for removal of eukaryotic cells/white blood cells from the sample. The kit further includes tertiary substrate for isolation of micro-organisms from the sample. The primary and secondary substrates are chosen from a group including CD45, CD15, CD4, CD3, and CD8. The tertiary protein is a pathogen binding protein. The matrix is a magnetic bead. Alternatively, the matrix may be a polystyrene bead. In an embodiment, the primary substrate coated onto one or more matrices is different from the secondary substrate. The primary and secondary substrates enable removal of white blood cells from the sample, thereby leaving behind micro-organisms unaffected. The tertiary substrate is then used to isolate the unaffected micro-organisms to obtain an enriched and concentrated sample of micro-organisms for further downstream processing.

The abovementioned method of enrichment of micro-organisms involves a combination of negative and positive enrichment methods. Negative enrichment provides elimination of non-target cells from the sample. Therefore, the non-target background is removed thereby providing a concentrated sample containing target cells. When followed by a positive enrichment act, the target cells are separated from any non-target cells that may have been left out in the process of negative enrichment. Therefore, the combination of negative and positive enrichment acts yields a highly enriched and concentrated sample of target cells, which are micro-organisms, in the present embodiment. The abovementioned method of enrichment of micro-organisms may be integrated efficiently with the downstream sample preparation methods. Library preparation acts such as dA tailing, adapter ligation is more efficient due to significant reduction in background human genomic DNA. The method also enables efficient amplification of whole genome of micro-organisms. A better ratio of microbial to human DNA sequences read allows for reduction in the time for genome sequencing. Thus, this enrichment method may be integrated with the sample preparation methods of any gene sequencing workflow that is used for metagenomics analysis of pathogens in a complex sample like whole blood.

The foregoing examples have been provided merely for the purpose of explanation and are in no way to be construed as limiting of the present disclosure. While the disclosure has been described with reference to various embodiments, it is understood that the words, which have been used herein, are words of description and illustration, rather than words of limitation. Further, although the disclosure has been described herein with reference to particular means, materials, and embodiments, the disclosure is not intended to be limited to the particulars disclosed herein; rather, the disclosure extends to all functionally equivalent structures, methods and uses, such as are within the scope of the appended claims. Those skilled in the art, having the benefit of the teachings of this specification, may effect numerous modifications thereto and changes may be made without departing from the scope and spirit of the disclosure in its aspects.

1. A method of enrichment of micro-organisms, in a metagenomics nucleic acid sequencing workflow, from a whole blood sample suspected to contain micro-organisms, the method comprising:
obtaining the whole blood sample suspected to contain micro-organisms, wherein the whole blood sample is obtained from a subject through a processing set;
concentrating the micro-organisms from the whole blood sample; and
processing the micro-organisms to obtain genetic information specific to the micro-organisms;
wherein, in the concentrating of the micro-organisms, the method further comprises:
separating plasma from the whole blood sample;
selectively removing white blood cells from the plasma; and
isolating the micro-organisms from the plasma after selectively removing the white blood cells,
wherein the micro-organisms are isolated from the plasma by incubating the plasma with at least one matrix coated with a tertiary substrate, and
wherein the tertiary substrate is histone proteins, methyl-CpG-binding domains, immune proteins, or combinations thereof.

2. The method of claim 1, wherein the separating of the plasma from the whole blood sample comprises usage of a centrifugation method.

3. The method of claim 2, wherein the whole blood sample is centrifuged at a relative centrifugal force ranging between 50×g and 500×g.

4. The method of claim 1, wherein the selectively removing of the white blood cells from the plasma comprises incubating the plasma with at least one matrix coated with a primary substrate having affinity for white blood cells.

5. The method of claim 4, wherein the selectively removing of the white blood cells from the plasma comprises incubating the plasma with at least one matrix coated with a secondary substrate having affinity for white blood cells, after usage of the primary substrate.

6. The method of claim 5, wherein the primary substrate and the secondary substrate and chosen from a group comprising antibodies against CD45, CD15, CD4, CD3, and CD8.

7. The method of claim 6, wherein the primary and secondary substrates are different from each other.

8. The method of claim 1, wherein the isolating of the micro-organisms from the plasma comprises incubating the plasma with at least one matrix coated with a tertiary substrate having affinity for the micro-organisms.

9. The method of claim 8, wherein the tertiary substrate is a pathogen binding protein.

10. The method of claim 4, wherein the at least one matrix is chosen from a group comprising of polystyrene beads and magnetic beads.

11. The method of claim 4, wherein the white blood cells bound to the at least one matrix are removed from the plasma using a magnet.

12. The method of claim 4, wherein the white blood cells bound to the at least one matrix are removed from the plasma using centrifugation.

13. A kit for carrying out an enrichment of micro-organisms in a metagenomics workflow, the kit comprising:
one or more first matrices coated with a primary substrate for removal of white blood cells, wherein the primary substrate has an affinity for white blood cells;
one or more second matrices coated with a secondary substrate for removal of white blood cells, wherein the secondary substrate has an affinity for white blood cells; and
one or more third matrices coated with a tertiary substrate for isolation of micro-organisms, wherein the tertiary substrate has an affinity for micro-organisms,
wherein the tertiary substrate is histone proteins, methyl-CpG-binding domains, immune proteins, or combinations thereof.

14. The kit of claim 13, wherein the primary substrate and secondary substrate are chosen from a group comprising antibodies against CD45, CD15, CD4, CD3, and CD8.

15. The kit of claim 13, wherein the tertiary substrate is a pathogen binding protein.

16. The kit of claim 13, wherein the one or more first matrices, the one or more second matrices, and the one or more third matrices are each chosen from a group comprising of polystyrene beads and magnetic beads.

17. A method of enrichment of micro-organisms, in a metagenomics nucleic acid sequencing workflow, from a whole blood sample suspected to contain micro-organisms, the method comprising:
providing a kit having: (1) one or more first matrices coated with a primary substrate for removal of white blood cells, wherein the primary substrate has an affinity for white blood cells; (2) one or more second matrices coated with a secondary substrate for removal of white blood cells, wherein the secondary substrate has an affinity for white blood cells; and (3) one or more third matrices coated with a tertiary substrate for isolation of micro-organisms, wherein the tertiary substrate has an affinity for micro-organisms, and wherein the tertiary substrate is histone proteins, methyl-CpG-binding domains, immune proteins, or combinations thereof;
obtaining the whole blood sample suspected to contain micro-organisms, wherein the whole blood sample is obtained from a subject through a processing set;
separating plasma from the whole blood sample;
selectively removing white blood cells from the plasma using the one or more first matrices and the one or more second matrices of the kit;
isolating the micro-organisms from the plasma after selectively removing the white blood cells by incubating the plasma with the one or more third matrices of the kit; and
processing the isolated micro-organisms to obtain genetic information specific to the micro-organisms.
